(19) United States
(12) Patent Application Publication
(10) Pub. No.: US 2015/0250992 A1
(43) Pub. Date: Sep. 10, 2015

(54) MECHANICAL DILATION OF THE OSTIA OF PARANASAL SINUSES AND OTHER PASSAGEWAYS OF THE EAR, NOSE AND THROAT

(71) Applicant: Acclarent, Inc., Menlo Park, CA (US)

(72) Inventors: John H. Morriss, San Francisco, CA (US); Thomas R. Jenkins, Alameda, CA (US); Eric Goldfarb, Belmont, CA (US); Scott J. Baron, Menlo Park, CA (US); Ketan P. Muni, San Jose, CA (US); John Y. Chang, Los Altos, CA (US); Julia D. Vrany, Los Altos, CA (US); Daniel T. Harfe, Los Altos, CA (US); Serena Swei Loh, San Carlos, CA (US); Robert N. Wood, Indian Beach, NC (US); Randy S. Chan, San Jose, CA (US); Hung V. Ha, San Jose, CA (US)

(21) Appl. No.: 14/658,432

(22) Filed: Mar. 16, 2015

Related U.S. Application Data
(63) Continuation of application No. 11/789,705, filed on Apr. 24, 2007, now abandoned.
Application No. 11/789,705 is a continuation-in-part of application No. 11/655,794, filed on Jan. 18, 2007, now Pat. No. 8,858,586.
Application No. 11/655,794 is a continuation-in-part of application No. 11/150,847, filed on Jun. 10, 2005, now Pat. No. 7,803,150.
Application No. 11/150,847 is a continuation-in-part of application No. 10/944,270, filed on Sep. 17, 2004, now abandoned.
Application No. 10/944,270 is a continuation-in-part of application No. 10/829,917, filed on Apr. 21, 2004, now Pat. No. 7,654,997.

(51) Int. Cl. A61M 29/02; A61B 17/24

(52) U.S. Cl. CPC A61M 29/02; A61B 17/24

(57) ABSTRACT

Devices and methods for dilating the ostia of paranasal sinuses and other passageways within the ear, nose and throat. A mechanical dilator is inserted through a nostril and used to dilate an ostium or a paranasal sinus or other passageway within the ear, nose or throat (e.g., a Eustachian tube or nasolacrimal duct). The mechanical dilator may be an expandable dilators that comprises one or more non-balloon structures with or without an accompanying balloon or a members of varying diameter or cross dimension (e.g., a tapered member) that may be advanced into the ostium or other passageway to cause dilation of that passageway.

### RELATED APPLICATIONS

This application is a continuation of U.S. patent application Ser. No. 11/789,705, entitled “Mechanical Dilation of the Ostia of Paranasal Sinuses and Other Passageways of the Ear, Nose and Throat,” filed on Apr. 24, 2007, which is a continuation in part of U.S. patent application Ser. No. 11/655,794, having an amended title of “Use of Mechanical Dilator Devices to Enlarge Ostia of Paranasal Sinuses and Other Passages in the Ear, Nose, Throat and Paranasal Sinuses” filed on Jan. 18, 2007 and issued as U.S. Pat. No. 8,858,586 on Oct. 14, 2014, which is a continuation in part of U.S. patent application Ser. No. 11/150,847, entitled “Devices, Systems and Methods Useable for Treating Sinusitis,” filed on Jun. 10, 2005 and issued as U.S. Pat. No. 7,803,150 on Sep. 28, 2010, which is a continuation in part of U.S. patent application Ser. No. 10/944,270, entitled “Apparatus and Methods for Dilating and Modifying Ostia of Paranasal Sinuses and Other Intranasal or Paranasal Structures,” filed on Sep. 17, 2004 (abandoned), which is a continuation in part of U.S. patent application Ser. No. 10/829,917, entitled “Devices, Systems and Methods for Diagnosing and Treating Sinusitis and Other Disorders of the Ears, Nose and/or Throat,” filed on Apr. 21, 2004 and issued as U.S. Pat. No. 7,654,997 on Feb. 2, 2010, the entire disclosures of such earlier filed applications being expressly incorporated herein by reference.

### FIELD OF THE INVENTION

The present invention relates generally to medical devices and methods and more particularly to devices and methods for dilating the ostia of paranasal sinuses and other passageways within the ear, nose and throat.

### BACKGROUND

The prior art has included a number of catheters that have radially expandable cages or other mechanically expandable dilators for expanding or compressing obstructions in blood vessels, the urethra other vessels. For example, U.S. Pat. No. 5,180,368 (Garrison) An intravascular catheter having an expandable cage mounted on the distal end of a tubular member which is radially expanded and contracted by means of a control wire. The control wire extends through a first inner lumen within the tubular member which extends along essentially the entire length thereof. A second inner lumen is provided in the distal portion of the tubular member which has a proximal guidewire port at least 15 cm but not more than 60 cm from the distal end of the catheter and a distal guidewire port which opens into the interior of the expandable cage. A guidewire or low profile fixed-wire steerable dilatation catheter is slidably disposed within the second lumen and a flexible tubular element extends through the expandable cage interior to facilitate the rapid exchange of the catheter. The catheter is particularly adapted to hold open an artery after a vascular procedure therein such as a balloon or other type of angioplasty.

U.S. Pat. No. 6,159,170 (Borodulin) describes a universal mechanical dilator combined with massaging action comprises a probe (12) consisting of a two rods (22 and 24), a drive unit (14) and an adapter (16) that connects the probe (12) to the drive unit. The instrument is intended for dilation of the urethra or other ducts of a human body and can operate in three different modes: pure dilation, pure vibration, and dilations combined with vibrations.

U.S. Pat. No. 6,059,752 (Segal) describes a mechanical dilatation and irradiation device for enlarging a flow passage of a vessel by dilating and irradiating an obstruction in the vessel. The device comprises a substantially cylindrically shaped expansion member and includes a means engaged to the expansion member for altering the distance between the proximal end and the distal end of the expansion member thereby transforming the expansion member between a diametrically contracted configuration to diametrically expanded configuration. A radioisotope is placed either inside the expansion member, alloyed into the metal from which the expansion member is constructed, coated onto the expansion member's exterior surface or alternately, the non-radioactive metal or alloy of the expansion member can be irradiated so that it has become radioactive. The radioactive expansion member or radioactive catheter is advanced through the vessel to the site of the obstruction and opposed axial forces are then applied to the expansion member causing it to expand, thereby dilating and irradiating the obstruction.

In recent years, new techniques have been developed for the treatment of sinusitis by inserting a dilation catheter into the nose, positioning the dilation catheter within the ostium of a paranasal sinus and using the dilator to expand the ostium, thereby improving drainage and ventilation of the paranasal sinus. These sinusitis treatment techniques may employ inflatable balloons or other dilators (i.e., expandable dilators that have mechanical component(s) rather than or in addition to a balloon). Some specific examples of the use of mechanical dilators for dilation of sinus ostia (or other passageways or ducts in the ear, nose or throat) are described in parent U.S. patent application Ser. Nos. 11/655,794, 11/150,847 and 10/944,270, which are incorporated herein by reference.

There remains a need for the further development of new mechanical dilator devices and methods for using mechanical dilator devices to dilate the ostia of paranasal sinuses and other passageways of the ear, nose or throat.

### SUMMARY OF THE INVENTION

In accordance with the invention, there are provided mechanical dilator devices and methods wherein mechanical dilators are used to dilate the ostia of paranasal sinuses or other passageways in the ear, nose or throat. As used in this patent application, the term “mechanical dilator” shall be interpreted to include at least; 1) expandable dilators that comprises one or more non-balloon dilation structures with or without an accompanying balloon and 2) members of varying diameter or cross dimension that may be advanced into a passageway to cause dilation of that passageway.

Further in accordance with the invention there are provided systems and methods for dilating the ostia of paranasal sinuses and other passageways in the ear, nose or throat of a human or animal subject by a) inserting a mechanical dilator through a nostril of the subject, positioning the mechanical dilator within the passageway and c) using the dilator to dilate the passageway. Ad described herein, the invention includes numerous types of mechanical dilators including but not limited to: Dilators That Shorten in a Longitudinal Dimension While Expanding in at Least One Transverse Dimension; Split Tube and Splayable Dilators; Dilators That Expand Transversely When Compressed Longitudinally; Dilators That Have Expandable Outer Covers With Apparatus Which Cause Expansion of the Outer Cover; Dilators That Expand Transversely In Response To Advancement or Retraction of a Wedge or Other Spreading Member; Dilators Having Hinged or Pivotal Members; Non-Expandable Tapered Dilators Dilators that Expand In Response to Conditions Within the Body and Mechanical Dilators In Combination With Balloons.

Still further embodiments, aspects, elements and details of the present invention will be understood upon reading of the detailed description and examples set forth herebelow.

### BRIEF DESCRIPTION OF THE DRAWINGS

FIG. 1 is a schematic showing of a human subject undergoing a procedure of the present invention for dilation of a passageway within the ear, nose or throat.

FIG. 1A is a partial sagittal sectional view of the head of the subject shown in FIG. 1.

FIGS. 2A and 2B show one embodiment of a dilator device of the present invention having an expandable dilator that shortens longitudinally as it expands.

FIGS. 2C and 2D show another embodiment of a dilator device of the present invention having an expandable dilator that shortens longitudinally as it expands.

FIGS. 2E and 2F show another embodiment of a dilator device of the present invention having an expandable dilator that shortens longitudinally as it expands.

FIGS. 2G and 2H show another embodiment of a dilator device of the present invention having an expandable dilator that shortens longitudinally as it expands.

FIGS. 2I and 2J show another embodiment of a dilator device of the present invention having an expandable dilator that shortens longitudinally as it expands.

FIGS. 2K and 2L show another embodiment of a dilator device of the present invention having an expandable dilator that shortens longitudinally as it expands.

FIGS. 2M and 2N show another embodiment of an expandable dilation member that shortens longitudinally as it expands and may be incorporated into a dilation device of the present invention.

FIGS. 3A and 3B show one embodiment of a dilation device of the present invention having an expandable dilator that comprises a plurality of members that expand outwardly.

FIG. 3C shows distal end views of the device of FIG. 3A in non-expanded (on left) and expanded (on right) configurations.

FIG. 3D is an enlarged perspective view of a distal portion of the device of FIG. 3A. FIGS. 3E and 3F show another embodiment of an expandable dilator comprising a split tube which forms individual outwardly expanding members.

FIGS. 3G and 3H show another embodiment of an expandable dilator comprising a split tube which forms individual outwardly expanding members.

FIGS. 4A and 4B show longitudinal sectional views of an embodiment of a dilator device of the present invention having a expandable dilator comprising a compressible material that expands transversely as it is compressed longitudinally.

FIGS. 5A and 5B show an embodiment of a dilator device of the present invention having an expandable dilator comprising a flexible or elastomeric outer cover and an apparatus within that outer cover which causes the outer cover to expand transversely.

FIG. 5C is a longitudinal sectional view of the distal portion of a dilator device of the type shown generally in FIGS. 5A and 5B, wherein the apparatus within the outer cover comprises a cam assembly disposed in a non-expanded position.

FIG. 5D is an enlarged cross sectional view of a portion of the outer cover of the device shown in FIG. 5C.

FIG. 5E is a showing of the device of FIG. 5C with the cam assembly disposed in an expanded configuration.

FIG. 5F is an exploded view of the cam assembly of the device shown in FIGS. 5C-5E.

FIGS. 6A and 6B show partial perspective views of an embodiment of a dilator device of the present invention having an expandable dilator that expands to a tapered shape.

FIG. 6C is a longitudinal sectional view through line 6C-6C of FIG. 6A.

FIG. 6D is a longitudinal sectional view through line 6D-6D of FIG. 6B.

FIGS. 7A and 7B are partial perspective views of another embodiment of a dilator device of the present invention having an expandable dilator comprising members that splay outwardly to a dilated configuration.

FIGS. 7C and 7D are partial longitudinal sectional views of another expandable dilator comprising members that splay outwardly to a dilated configuration.

FIGS. 7E and 7F are partial longitudinal sectional views of another expandable dilator comprising members that splay outwardly to a dilated configuration.

FIGS. 7G and 7H are partial longitudinal sectional views of another expandable dilator comprising members that splay outwardly to a dilated configuration.

FIG. 7I is a side view of another embodiment of a dilator device of the present invention having an expandable dilator comprising members that splay outwardly to a dilated configuration.

FIG. 7J is a longitudinal section view through the expandable dilation of the device of FIG. 7I while the dilator is in an expanded configuration.

FIGS. 8A and 8B show schematic diagrams of an embodiment of a dilation device of the present invention having an expandable dilator that comprises hinged members.

FIGS. 8C and 8D show the dilation device of FIGS. 8A and 8B transitioning to an expanded configuration.

FIGS. 8E and 8F are schematic diagrams of an embodiment of a dilation device of the present invention having an expandable dilator that comprises pivotally connected members.

FIGS. 8G and 8H show schematic diagrams of an embodiment of a dilation device of the present invention having an expandable dilator that comprises pivotally connected members.

FIGS. 9A and 9B are partial side views of alternative dilator devices of the present invention.

FIGS. 9C and 9D are partial side views of another dilator device of the present invention wherein the dilator comprises a tapered surface with an everting cover.

FIGS. 9E and 9F are partial side views of another dilator device of the present invention wherein the dilator comprises a tapered surface mounted distal to a telescoping shaft segment.

FIGS. 9G and 9H are partial side views of another dilator device of the present invention wherein the dilator comprises a region on the distal end of the device that is initially tapered to a narrow configuration and subsequently expands to an expanded configuration when a mandrel is advanced therethrough.

FIG. 9I shows another device of the present invention comprising a rigid or malleable shaft with a dilator bulb formed at its distal end.

FIG. 9J shows a curved version of the device of FIG. 91.

FIGS. 10A and 10B show another dilator device of the present invention having an expandable dilator that comprises a structure that expands as it warms to body temperature.

FIG. 11 is a broken, longitudinal sectional view of another dilation device of the present invention having an expandable dilator comprising a flexible or elastomeric cover within which a filamentous or similar bulking material accumulates to cause expansion of the dilator.

FIGS. 11A and 11B show different embodiments of bulking material configurations.

FIG. 12A is a partial right longitudinal sectional view of a mechanical dilator device having a balloon in addition to the mechanical dilator.

FIG. 12B is a partial left longitudinal sectional view of the device of FIG. 12A.

### DETAILED DESCRIPTION

The following detailed description, the accompanying drawings are intended to describe some, but not necessarily all, examples or embodiments of the invention. The contents of this detailed description do not limit the scope of the invention in any way.

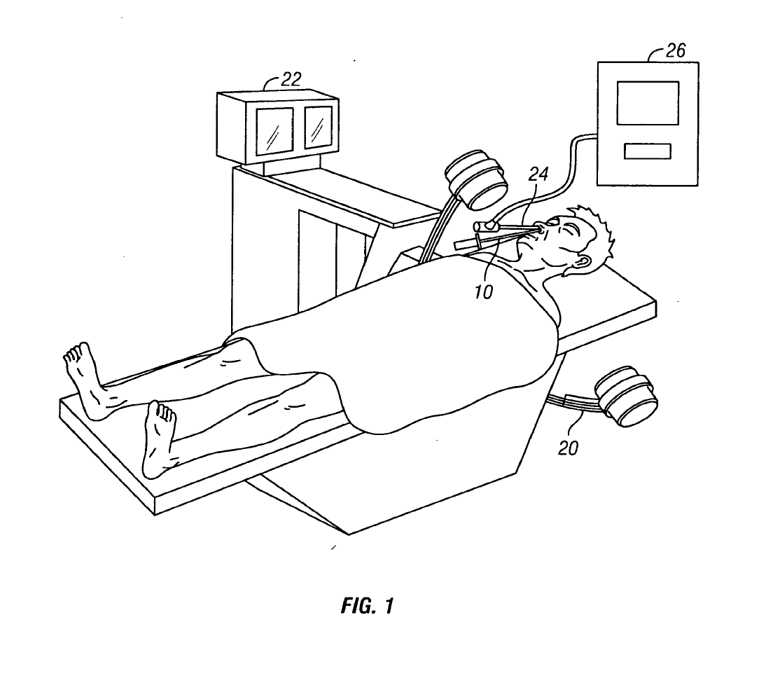

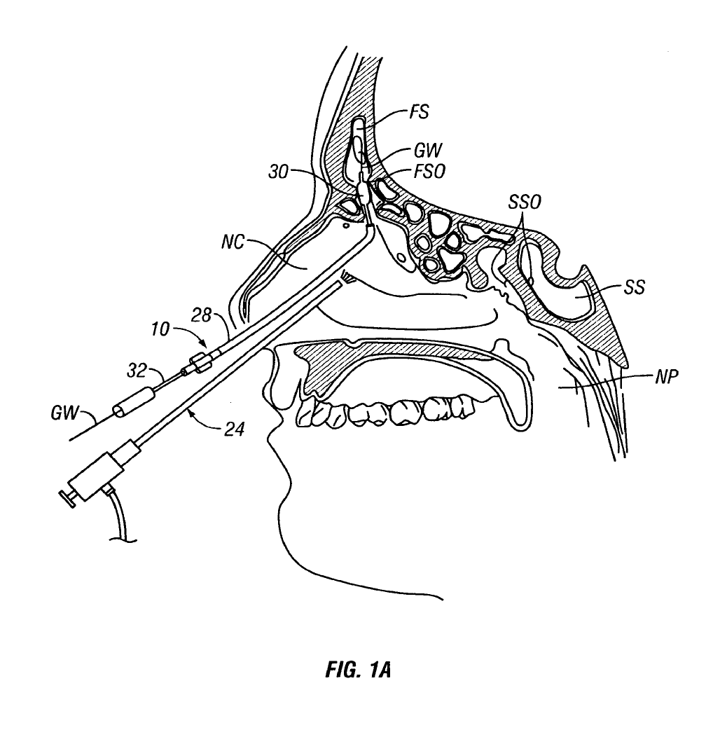

FIG. 1 shows a human subject undergoing a procedure wherein a trans-nasally inserted mechanical dilation device 10 is used to dilate an opening of a paranasal sinus or other passageway within the ear, nose or throat. As shown, an endoscope 24 may optionally be inserted into the subject's nose and used to view some or all of the procedure on an endoscope monitor 26. Also, optionally, an imaging device such as a C-arm fluoroscope 20 and monitor 22 (or any other suitable imaging device such as a stationary fluoroscope, fluoro-CT scanner, portable or intraoperative CT scanner, etc.) may also be used to obtain images of the device(s) and/or relevant anatomy during some or all of the procedure. To facilitate this, as those of skill in the art will appreciate, endoscopically visible and/or radiographically visible markers may be provided on any of the devices of the present invention at locations which will facilitate use of the endoscope to assist the operator in positioning and/or using the devices in a safe and effective manner.

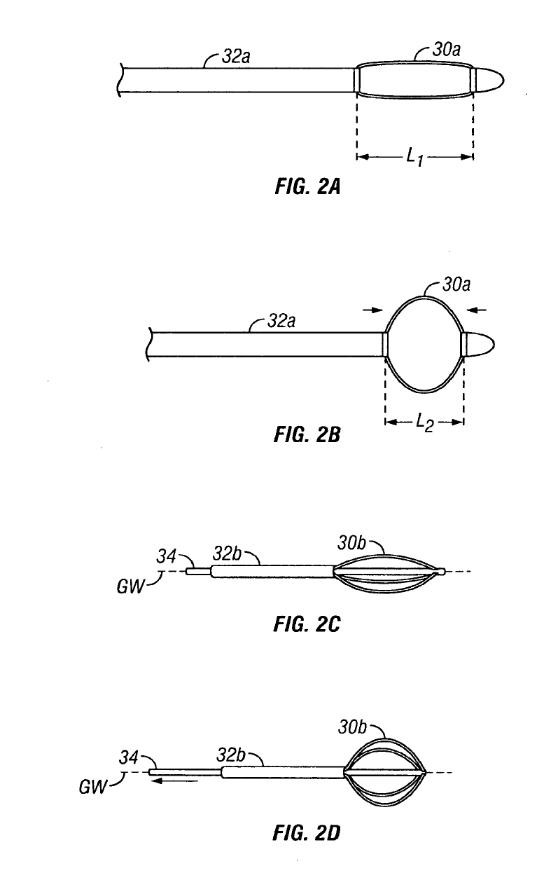

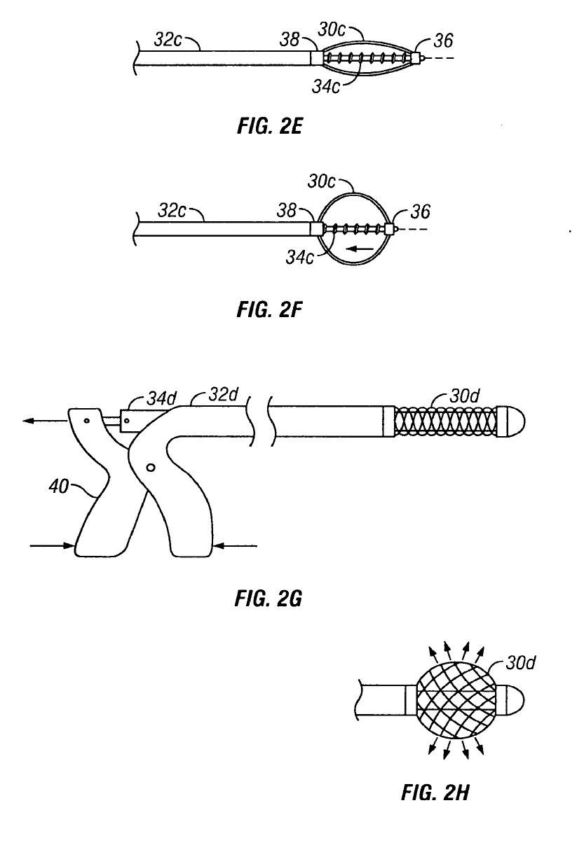

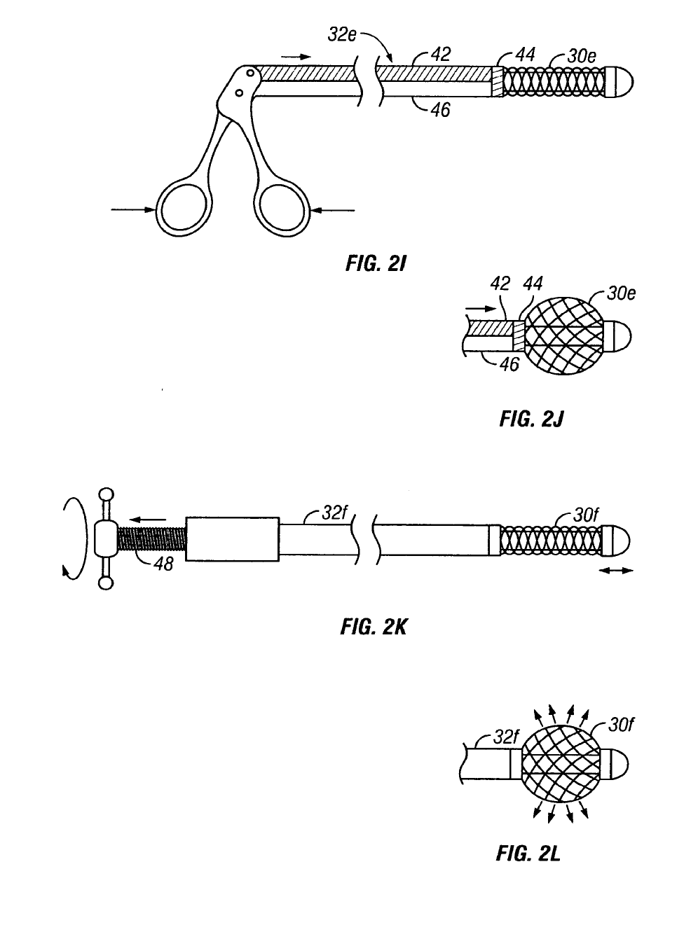

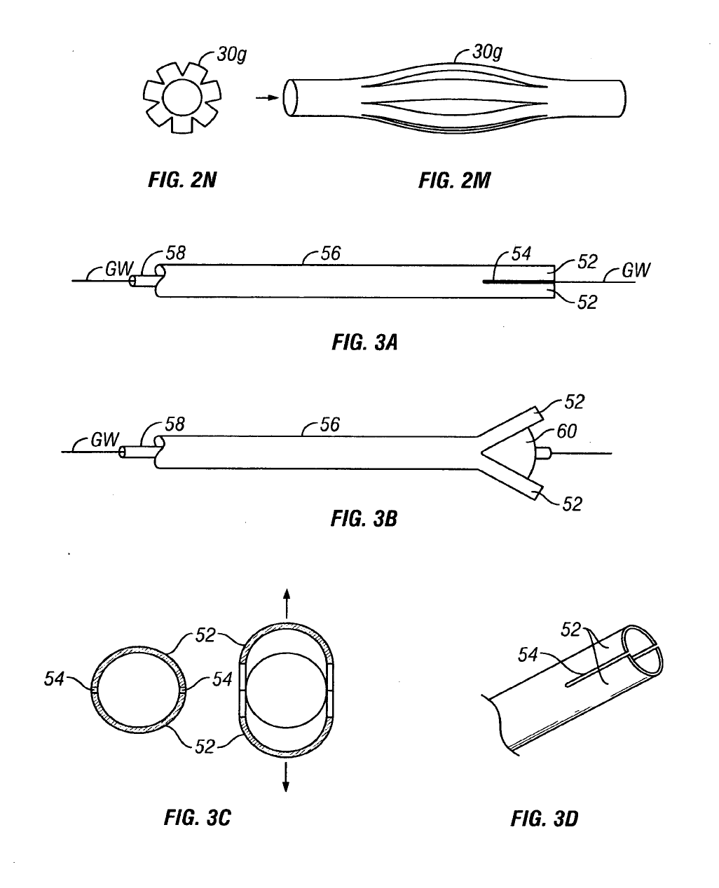

FIG. 2 is a general showing of one example of a method of the present invention wherein the dilation device 10 comprises a dilator 30 on an elongate shaft 32 through which a guidewire GW is inserted and which is insertable through a guide catheter 28. Examples of guide catheters through which dilator devices of the present invention may be inserted, where appropriate, include those described in parent application Ser. Nos. 11/655,794; 11/150,847 and 10/944,270, which are incorporated herein by reference. Also, trans-nasal guide catheters useable for guiding some of the dilator devices of the present invention are available commercially as Relieva sinus guide catheters from Acclarent, Inc., Menlo park, California. In the showing of FIG. 2, an optional endoscope 24 is inserted into the nostril and is being used to observe the insertion and positioning of the dilation device 10 and/or the guide catheter 28. In this example, the guide catheter 28 was initially inserted to a location near the frontal sinus ostium FSO and the guidewire GW was then advanced into the frontal sinus FS. Thereafter, a guidewire lumen formed in the dilator device shaft 32 was used to advance the dilator device over the guidewire GW and through the guide catheter 28 to a position where the dilator 30 is within the frontal sinus ostium FSO and is thus used to dilate that frontal sinus ostium FSO to improve drainage and ventilation of the frontal sinus FS. This is but one of many examples of the manner in which the devices of the present invention may be used. The dilation devices of the present invention may be used to dilate the natural or surgically modified ostia of any paranasal sinuses (i.e., frontal, maxillary, sphenoid, ethmoid or frontal) and/or other passageways of the ear, nose or throat, such as the Eustachian tubes or the naso-lacrimal ducts.

FIGS. 2A through 11 show various non-limiting examples of mechanical dilator devices that may be used to dilate passageways of the ear, nose and throat. These examples include expandable dilators which may be advanced into the desired passageway while in a non-expanded state and then expanded to cause dilation of the passageway as well as dilators that have regions of varying diameter or transverse dimension (e.g., tapered surfaces) that may be advanced into the passageway so as to dilate the passageway.

### Dilators that Shorten in a Longitudinal Dimension while Expanding in at Least One Transverse Dimension

FIGS. 2A through 2M show several examples of expandable mechanical dilators which shorten longitudinally as they expand transversely.

FIGS. 2A and 2B show one example of a dilator device having an expandable dilator 30a on an elongate shaft 32a. The dilator 30a may comprise a generally cylindrical member formed of solid or substantially continuous material (e.g., polymeric elastomeric tubing, film, sheet, woven Dacron, etc.) or non-solid or non-continuous material (mesh, screen, a wire cage, spaced apart struts, a single strut, etc.). The shaft 32a may be rigid, flexible or malleable and may or may not include a guidewire lumen or other working lumens. One or more actuating members (pull wire(s), cable(s), push/pull shaft(s), screw(s), rotatable rods, inner shaft members, etc.) extend through the shaft 32a and is/are useable to cause the dilator 30a to shorten from its initial non-expanded length L₁ to its expanded length L2 thereby causing concurrent transverse expansion of the dilator 30a, as shown. More specific examples of this generic mechanical dilation device as seen in FIGS. 2C-2M.

FIGS. 2C and 2D show a dilation device comprising an expandable dilator 30b having a plurality of strut members 31 at radially spaced apart locations with their proximal ends attached to the distal end of an outer shaft member 32b and their distal ends attached to the distal end of an inner shaft member 34. These strut members 31 may be formed of plastic, metal or any suitable material and may be in the form of wires, ribbons, strands, etc. A guidewire GW extends through a lumen in the inner shaft member 34. When the outer shaft member 32B is in a retracted position as seen in FIG. 2C, the dilator 30b will be in a non-expanded configuration. When the outer shaft member 32b is advanced in the distal direction, it will push the proximal ends of the strut members forward, thereby causing the strut members 31 to bow outwardly and effectively causing the dilator 30b to shorten longitudinally, as shown in FIG. 2D. Thereafter, when the outer shaft member 30b is retracted in the proximal direction, the dilator will again elongate and the strut members 31 will return to their non-expanded configurations. Although a total of four strut members 31 are shown in this example, it is to be appreciated that various numbers of strut members 312 may be used and, for some applications, even a single strut member 31 may be suitable.

FIGS. 2E and 2F show an embodiment of a dilation device comprising an expandable dilator 30c on the end of a shaft 32c. The dilator 30c comprises two strut members 33 at diametrically opposite positions with their distal ends anchored to a distal structure 36 and their proximal ends anchored to a proximal structure 38 on the distal end of the shaft 32c. A screw member 34c extends through the shaft 32c. When the screw member is rotated in a particular direction, it causes the distal structure 36 to move in the proximal direction, toward the proximal structure 38, thereby causing longitudinal shortening of the dilator 30c and outward bowing of the strut members 33, as seen in FIG. 2F. Rotation of the screw member 34c in the other direction will cause the dilator 30c to return to its non-expanded configuration shown in FIG. 2E.

FIGS. 2G and 2H show an example of a dilator device having a dilator 30d that comprises a cylindrical mesh tube (e.g., wire or plastic mesh). The distal end of the mesh tube is secured to a tip member on the end of an inner shaft member 34d and the proximal end of the mesh tube is secured to the distal end of an outer shaft member. As with any devices of the present invention, a suitable handpiece may be provided to facilitate handling and actuation of the device. In this example, the handpiece comprises a pistol grip handpiece 40 which, when squeezed, causes the outer shaft to move in the distal direction and/or the inner shaft member 34d to move in the proximal direction. This results in longitudinal shortening and outward radial expansion of the dilator 30d, as seen in FIG. 2H. The handpiece 40 may be biased to the non-expanded position such that, when the operator stops squeezing the handpiece 40, the outer shaft member 32d will automatically retract in the proximal direction and the dilator 30d will return to its non-expanded configuration as seen in FIG. 2G.

FIGS. 2I and 2J show an embodiment of a dilator device having a dilator 30e that may comprise the same type of mesh tube as described above with respect to FIGS. 2G and 2H. However, in this embodiment, there is a stationary lower shaft member 46 and a moveable upper shaft member 42. The distal end of the mesh tube is secured to a tip member on the distal end of the stationary lower shaft member 46. The proximal end of the mesh tube is secured to a ring member 44 on the distal end of the longitudinally moveable upper shaft member 42. This ring member 44 extends around the stationary lower shaft member 46, as shown. The handpiece in this embodiment is a scissor grip handpiece 48. When the operator squeezes the scissor grip handpiece 48, the upper shaft member 42 and ring 44 move in the distal direction. This results in longitudinal shortening and outward radial expansion of the dilator 30e, as seen in FIG. 2J. When the jaws of the handpeice 48 are again moved apart, the upper shaft member 42 and ring 44 will retract in the proximal direction and the dilator 30E will return to its non-expanded configuration as seen in FIG. 21.

FIGS. 2K and 2L show yet another embodiment of a dilator device. In this embodiment, the dilator 30f may comprise the same type of mesh tube as described above with respect to FIGS. 2G and 2H. However, in this embodiment, there is a stationary outer shaft 32f and a longitudinally advanceable and retractable inner shaft 48 that extends coaxially through the outer shaft 32f. A proximal region of the inner shaft 48 is externally threaded and mated with an internally threaded region on the outer shaft 32f (or on another structure) such that the inner shaft 48 may be advanced distally by rotating it in one direction and retracted proximally by rotating it in the other direction. The distal end of the mesh tube is secured to the distal end of the inner shaft 48 and the proximal end of the mesh tube is secured to the distal end of the outer shaft 32f. When the inner shaft 48 is rotated in the first direction, it will advance distally causing the dilator 30f to shorten longitudinally and expand radially, as seen in FIG. 2L. When the inner shaft 48 is rotated in the other direction, it will retract proximally causing the dilator 30f to lengthen longitudinally and contract radially, as seen in FIG. 2K.

It is to be appreciated that the mesh tube dilators 30d, 30e and 30f seen in FIGS. 2G-2L, the strut dilators 30b and 30c seen in FIGS. 2C-2F and the other types of dilators referred to above (e.g., solid or substantially continuous material such as polymeric elastomeric tubing, film, sheet, woven Dacron, etc. or non-solid or non-continuous materials such as mesh, screen, wire cages, spaced apart struts, single struts, etc. are merely examples and the invention is not limited to dilations of those types of construction. By way of further example, FIGS. 2M and 2N show another dilator 30g that may be used on any of the devices illustrated in FIGS. 2A-2L. This dilator 2M comprises a segment of polymeric tube having longitudinal slits formed at radially spaced apart locations about the mid-region of the tube. When either end of this tube is forced toward the other, the tube will longitudinally shorten and the ribbon like strips of tube between the lists will bow outwardly to a radially expanded configuration as seen in FIGS. 2N and 2M.

### Split Tube and Splayable Dilators

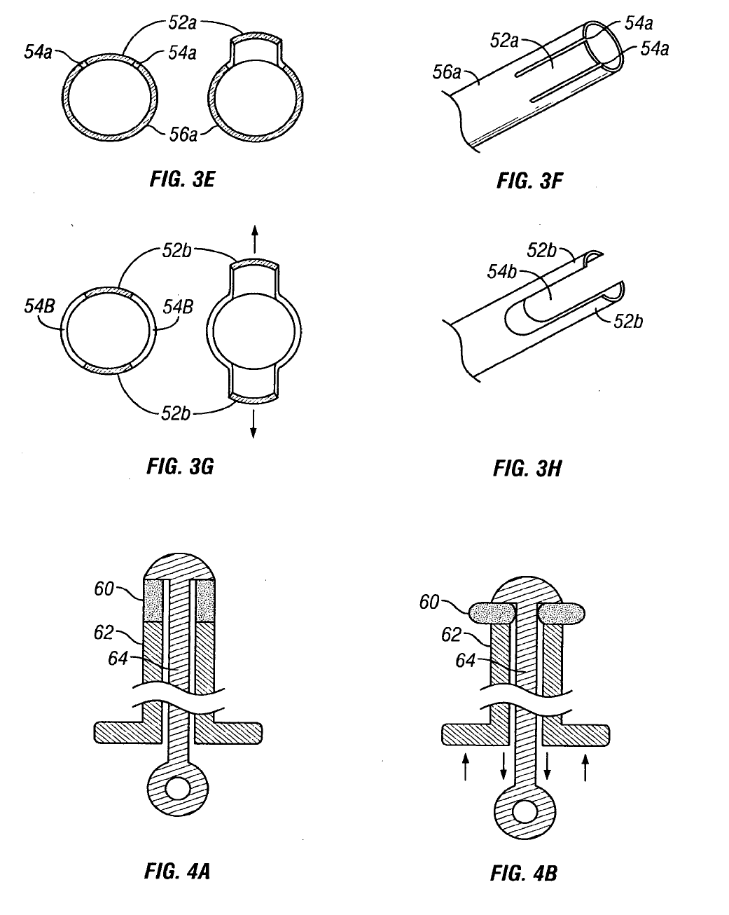

FIGS. 3A-3H show examples of dilation devices that comprise tubular shafts having longitudinal slits formed in the distal end of the tube to create one or more individual members that splay or expand outwardly to dilate the ostium or other passageway in which the dilator is positioned. For example, FIGS. 3A-3D show one embodiment of a dilation device comprising a tubular shaft 56 having a longitudinal slit 54 that extends from the distal end of the tubular shaft 56 at a location that is approximately 3 to 9 mm proximal to the distal end of the tubular shaft 56, thereby creating separable members 52 at the distal end of the tubular shaft 56. In this example, an expander such as a balloon catheter 58 extends though the tubular shaft 56 with its balloon 60 positioned within the distal portion of the tubular shaft 56 between the separable members 52. The balloon catheter 58 may optionally have a guidewire lumen to allow the device to be advanced over a guidewire GW, as shown. After the distal end of the tubular shaft 56 has been maneuvered into position within the ostium or passageway to be dilated, the balloon 60 is inflated, causing the members 52 to splay apart in diametrically opposite directions, thereby dilating the passageway in that plane. This device may be used to effect dilation of a passageway in only one plane. Or, this device may be rotated within the passageway and re-expanded, one or more times, to effect expansion of the passageway in a plurality of planes or, potentially, to effect 360 degree radial expansion of the passageway.

The specific plane or direction in which the passageway is dilated may be controlled by changing the number, width and/or location of the slit(s) 54 formed in the distal portion of the tubular shaft 56 to form a single member 52a between those slits. For example, FIGS. 3E and 3F show a device where two slits 54a are formed closer together than in the embodiment of FIGS. 3A-3D. An expander such as a specially configured balloon or spring wire that bows in the direction of the member 52a will cause the member 52a to splay outwardly as seen in the right side of FIG. 3E. Thus, this device could be used to exert concentrated force on the wall of the passageway in a single direction, thereby dilating the passageway to one side but not the other. By way of another example, FIGS. 3G and 3H show another embodiment of an expandable dilator comprising a tubular shaft 56b with wide slits 54b formed on opposite sides of the distal portion of the tubular shaft 56b, thereby forming two relatively narrow members 52b at diametrically spaced apart locations. An expander, such as the balloon catheter 58 described above, may be positioned within the distal portion of the tubular shaft 56b between the members 52b and may be used to cause the members 52b to splay outwardly, thereby widening the ostium or passageway in a relatively narrow plane.

### Dilators that Expand Transversely when Compressed Longitudinally

FIGS. 4A and 4B show longitudinal sectional views of an embodiment of a dilator device of the present invention having a expandable dilator having a compressible dilator 60 formed of an elastomeric material, gel filled bag or similar compressible material that will radially expand when longitudinally compressed. The proximal end of the dilator 60 abuts against the distal end of a tubular shaft 62 and the distal end of the dilator 60 abuts against a flange on the end of a plunger 64 that extends through the tubular shaft 62 and through the dilator 60, as shown. After the device has been inserted into the subject's body and positioned such that the dilator 60 is within the ostium or passageway to be dilated, the plunger 64 is withdrawn in the proximal direction and/or the tubular shaft 62 is advanced in the distal direction, thereby longitudinally compressing the dilator 60 and causing the dilator 60 to expand radially as seen in FIG. 4B, thereby resulting in dilation of the ostium or other passageway. After the ostium or other passageway has been dilated, the plunger 64 is advanced in the distal direction and/or the tubular shaft 62 is retracted in the proximal direction, thereby allowing the dilator 60 to elongate longitudinally and contract radially to its original configuration, as shown in FIG. 4A.

A method according to claim 3 wherein the expandable dilator comprises an expandable outer cover and expansion apparatus useable to cause the outer cover to expand when desired.

### Dilators that have Expandable Outer Covers with Apparatus which Cause Expansion of the Outer Cover

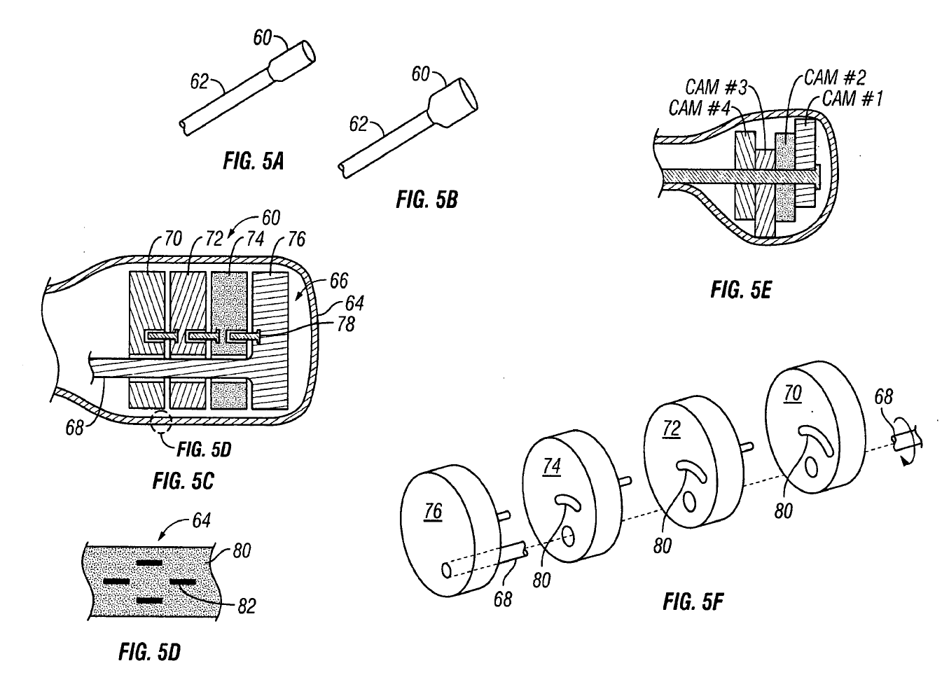

The present invention also include dilators that have full or partial expandable outer covers with apparatus for causing the outer cover to expand in a transverse dimension after the dilator has been positioned within the ostium or other passageway to be dilated. As explained more fully below, the outer cover may, in some cases, be constructed to distribute or disseminate the force exerted on the adjacent tissue as the dilator expands. For example, FIGS. 5A-5F show a dilator device 62 having an elongate shaft 62 and an expandable dilator 60 positioned on the shaft 62. The shaft 62 may be flexible, rigid or malleable. As may be appreciated from the showings of FIGS. 5C-5F, in this example the dilator 60 comprises an expandable outer cover 64 having a cam assembly 66 disposed therein. The can assembly is initially disposed in a non-expanded configuration as seen in FIG. 5C and is transitionable to an expanded configuration as seen in FIG. 5E. The outer cover 64 is formed of an elastomeric material 80 that has reinforcing members 82 (e.g., fibers, filaments, wires, metal strips, braid, woven fiber material, etc.) extending through the elastomeric material 80 to impart desired rigidity to at least the side walls of the outer cover 64 so that the side walls will remain substantially flat and will not assume a bumpy or wavy configuration conforming directly to the shape of the underlying cam surfaces when in the expanded configuration seen in FIG. 5E. In this manner the cover 64 expands radially and distributes the expansion force evenly over the adjacent tissue of the anatomical ostium or other passageway being dilated.

The cam assembly comprises first, second, third and fourth cams 70, 72, 74, 76 mounted on an off-center drive shaft 68. Only the fourth cam 76 is directly connected to the drive shaft 68 such that rotation of the drive shaft 68 causes concurrent rotation of only the fourth cam 76. The first, second and third cams 70, 72, 74 have arcuate slots of varying length formed in the distal sides thereof and the second, third and fourth cams 72, 74, 76 have pins 78 that extend from the proximal sides thereof and insert into the slots 80 on the neighboring cams, as may be appreciated from FIGS. 5C and 5F. When it is desired to cause the dilator 60 to expand, the drive shaft 68 is rotated, causing corresponding rotation of the fourth cam 76, the pin 78 on the fourth cam 76 advances through the slot 80 on the neighboring third cam 74 until it reaches the end of that slot 80 at which time the pin 78 of the fourth cam 76 begins to drive rotation of the third cam 74. The pin 78 on the third cam 74 advances through the slot 80 on the neighboring second cam 72 until it reaches the end of that slot 80 at which time the pin 78 of the third cam 74 begins to drive rotation of the second cam 73. The pin 78 on the second cam 72 advances through the slot 80 on the neighboring first cam 70 until it reaches the end of that slot 80 at which time the pin 78 of the second cam 72 begins to drive rotation of the first cam 70. Because the drive shaft 68 extends through the cams 70, 72, 74, 76 in an off-center fashion, this results in an enlargement of the overall width of the cam assembly as seen in FIG. 5E, thereby expanding the outer cover 64. When it is desired to return the dilator 60 to its non-expanded configuration, the drive shaft 68 is rotated in the opposite direction, causing the cams 70, 72, 74, 76 to once again assume the non-expanded configuration seen in FIG. 5C.

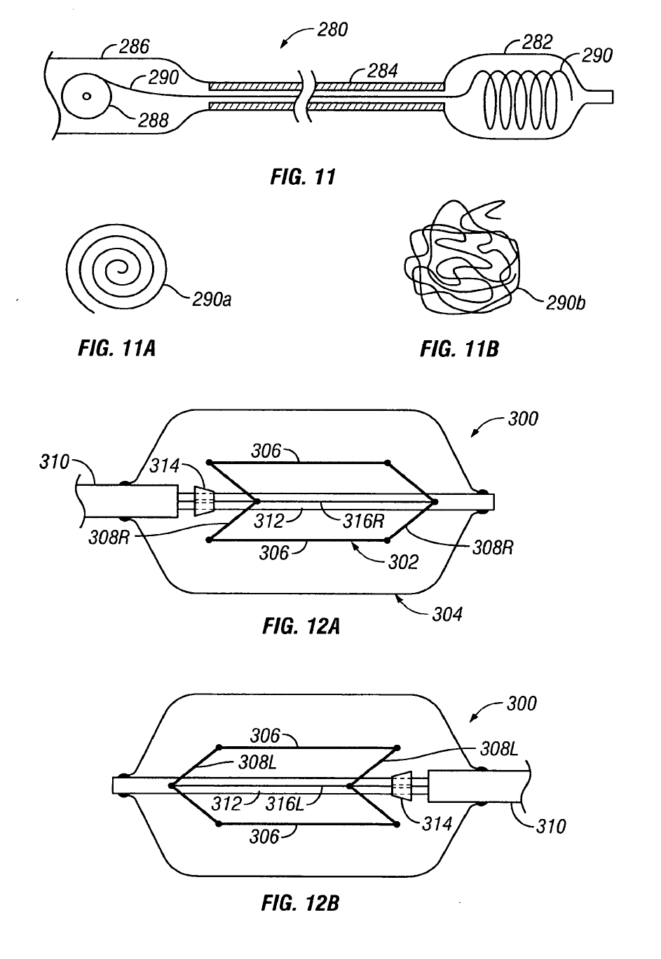

Another dilator having an expandable outer cover is shown in FIG. 11. This dilator 280 comprises an elongate tubular shaft 284 having a handpiece 286 on its proximal end and a dilator comprising an expandable outer cover 282 disposed at or near the distal end of the shaft 284. A reel 288 is mounted in the handpiece 286. A member 290 such as a filament, wire, fiber, etc. is initially wound onto the reel 288 and the member 290 extends through tubular shaft 284 into a cavity within the expandable cover 282.

When it is desired to expand the dilator, the reel 288 turns in a direction which feeds the member 290 into the cavity within the expandable cover 282, thereby causing the outer cover 282 to expand transversely. When it is subsequently desired to return the dilator to its non-expanded configuration, the reel 288 is rotated in the opposite direction, thereby retracting the member 290 out of the cavity within the expandable cover 282 and back into the handpiece 286. In some embodiments, the member 290 may be biased to a specific shape, such as the spiral or watch spring like configuration of the member 290a shown in FIG. 11A. In other embodiments, the member 290 may form a random coil or may otherwise collect in random fashion within the expandable cover 282, as demonstrated by the member 290b shown in FIG. 11B.

Another type or apparatus that may be used to expand an outer cover or other expandable structure described herein is a magnetic apparatus where magnets are initially positioned adjacent to one another (e.g., side by side or one in front of the other) such that their magnetic fields do not substantially repel one another. When it is desired to expand the dilator, a pull member may be used to pull one of the magnets to a position on top of, below or next to the other such that opposite poles of the magnets are juxtaposed and the magnetic force forces the magnets apart, thereby expanding the dilator. When it is desired to return the dilator to its non-expanded state a push member may be used to pus the first magnet back to its original position.

Another type or apparatus that may be used to expand an outer cover or other expandable structure described herein is a vibrator. A small vibrator, such as an off center motor, could be positioned within the cover. Prior to energization of the vibrator, the device would be in a non-expanded state. However, after energization of the vibrator, vibratory movement of the vibrator will cause the expandable cover to expand.

### Dilators that Expand Transversely in Response to Advancement or Retraction of a Wedge or Other Spreading Member

The invention also includes a number of dilator devices having expandable dilators that undergo expansion when a wedge or other spreading member is advanced or retracted between portions of the dilator, causing them to separate and expand outwardly.

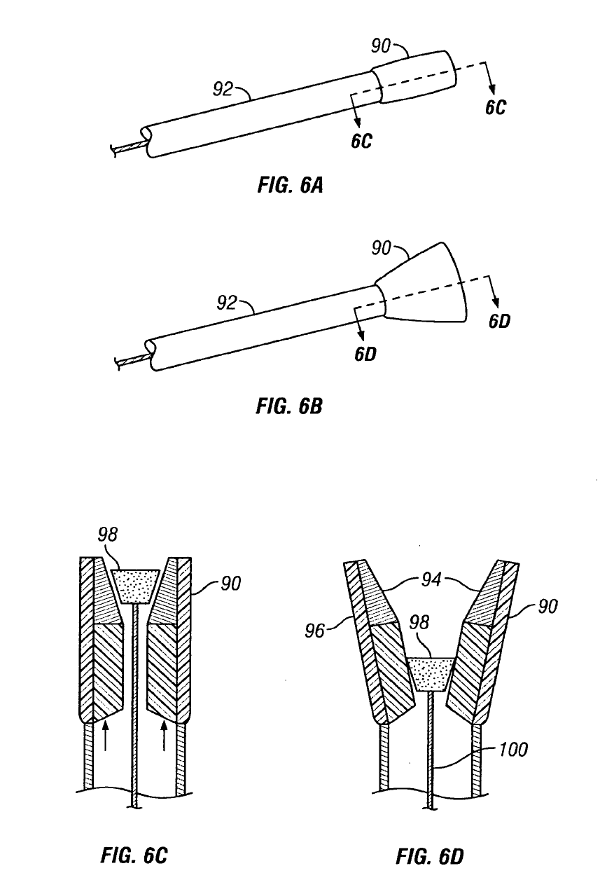

For example, FIGS. 6A-6D show a dilation device that comprises an elongate shaft 92 having an expandable dilator 90 mounted on the elongate shaft 92. The shaft 92 may be flexible, rigid or malleable. The dilator comprises a generally cylindrical, expandable elastomeric tube having an inner lumen with an enlarged tapered distal region 94. A round, tapered wedge member 98 is connected to an inner pull member 100 such as a wire or other elongate member. Initially, while the dilator 90 is in its non-expanded configuration, the wedge member 98 resides within the enlarged tapered distal region 94 of the dilator lumen, as seen in FIG. 6C. Thereafter, when it is desired to expand the dilator 90, the pull member 100 is pulled in the proximal direction causing the wedge member to be retracted within the lumen of the dilator 90, thereby causing the distal portion of the dilation to expand radially. This results in the dilator assuming a tapered expanded configuration, as seen in FIGS. 6B and 6D. The tapered expanded configuration may be useful when expanding passageways that are naturally tapered or where it is intended to dilate one region of the passageway more than another region.

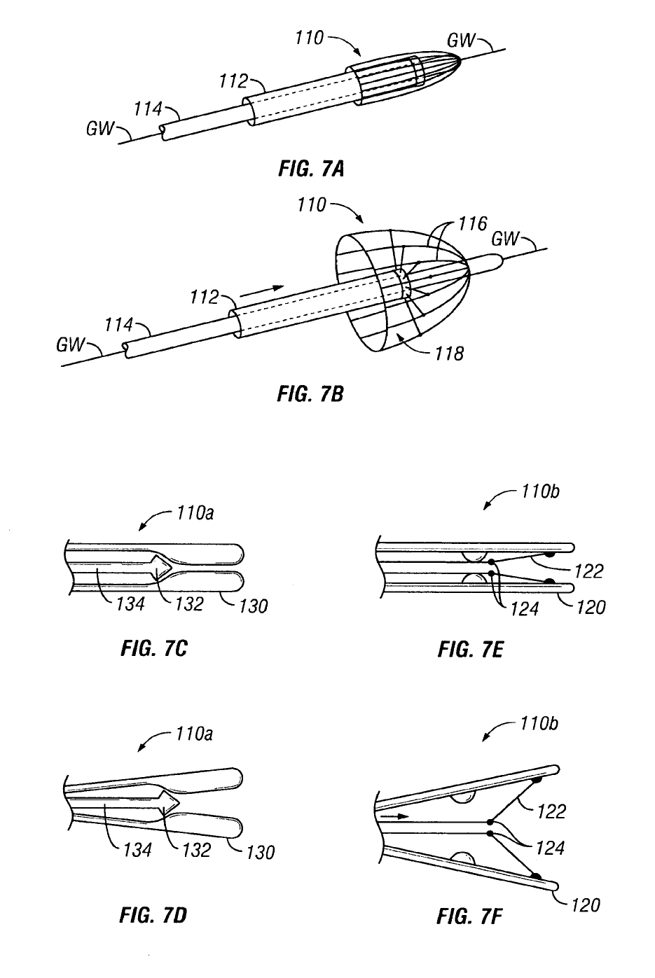

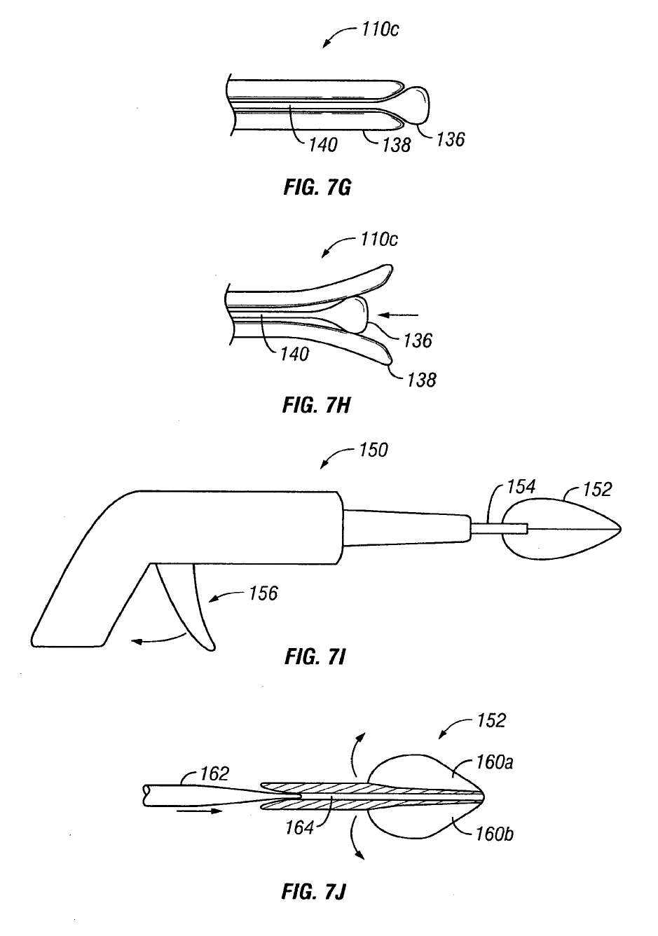

Also, FIGS. 7D and 7F show a dilator 110a that may be positioned on the distal end of an elongate flexible, rigid or malleable shaft. This dilator 110a comprises generally cylindrical, expandable elastomeric tube having an inner lumen with a narrowed distal region, as shown. While the dilator 110a is in its non-expanded configuration, a push member 134 having a spreading member 132 on its distal end is initially disposed within the lumen of the dilator 110a proximal to the narrowed distal region, as seen in FIG. 7C. When it is desired to expand the dilator 110a, the push member 134 is advanced in the distal direction causing the spreading member 132 to be pushed into the narrowed distal region of the dilator lumen, thereby causing the distal end of the dilator 110a to expand as seen in FIG. 7D.

FIGS. 7G and 7H show another dilator 110c that may be mounted on the distal end of an elongate flexible, rigid or malleable shaft. This dilator 110c comprises generally cylindrical, expandable elastomeric tube having an inner lumen extending therethrough. While the dilator 110c is in its non-expanded configuration, a pull member 140 having a spreading member 138 on its distal end is initially disposed within the lumen of the dilator 110c with the spreading member 136 located distal to the lumen of the dilator 110c, as seen in FIG. 7G. When it is desired to expand the dilator 110c, the pull member 140 is retracted in the proximal direction causing the spreading member 136 to be pulled into the dilator lumen, thereby causing the distal end of the dilator 110c to expand as seen in FIG. 7H.

FIGS. 7I and 7J show a dilation device 150 having another dilator 152 located on the distal end of an elongate shaft 154. In this example, the elongate shaft 154 comprises a rigid shaft that extends from a pistol grip handpiece 156 of a type well known in the art of surgical and interventional medical devices. The dilator 152 comprises an ovoid body that is divided into a first portion 160a and a second portion 160b. While the dilator 152 is in its non-expanded configuration, a tapered wedge member 162 is positioned within the shaft 154 proximal to the dilator 152. When it is desired to expand the dilator 152, the operator squeezes the trigger of the pistol grip handpiece 156 which in turn causes the wedge member 164 to advance to a position between the first portion 160a and second portion 160b of the dilator 152, thereby spreading the first portion 160a and second portion 160b way from each other and resulting in transverse expansion of the dilator 152, as seen in FIG. 7J.

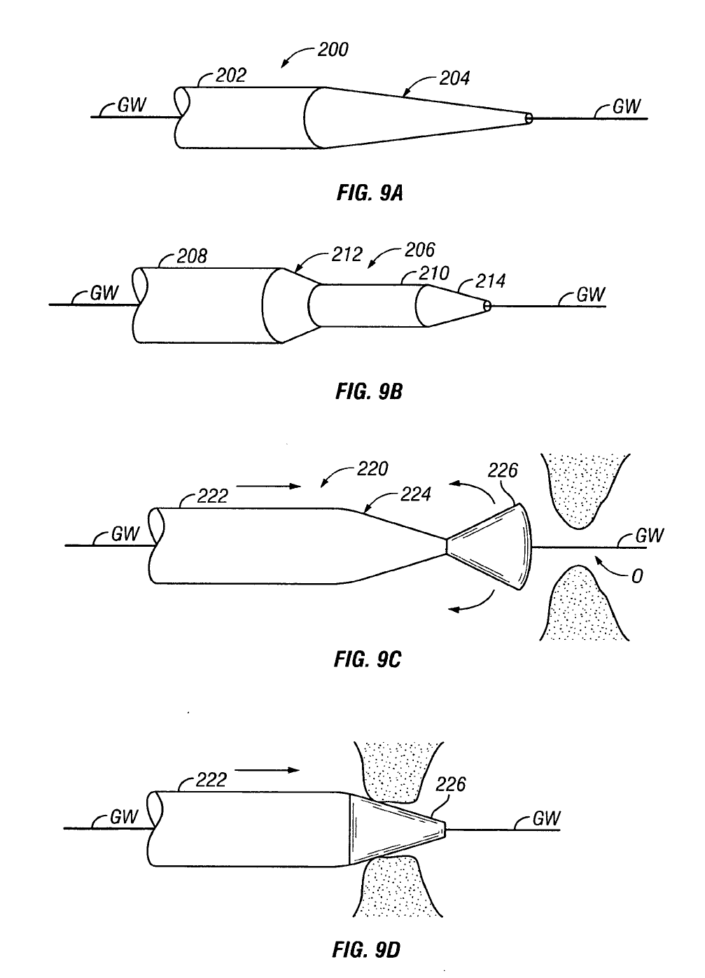

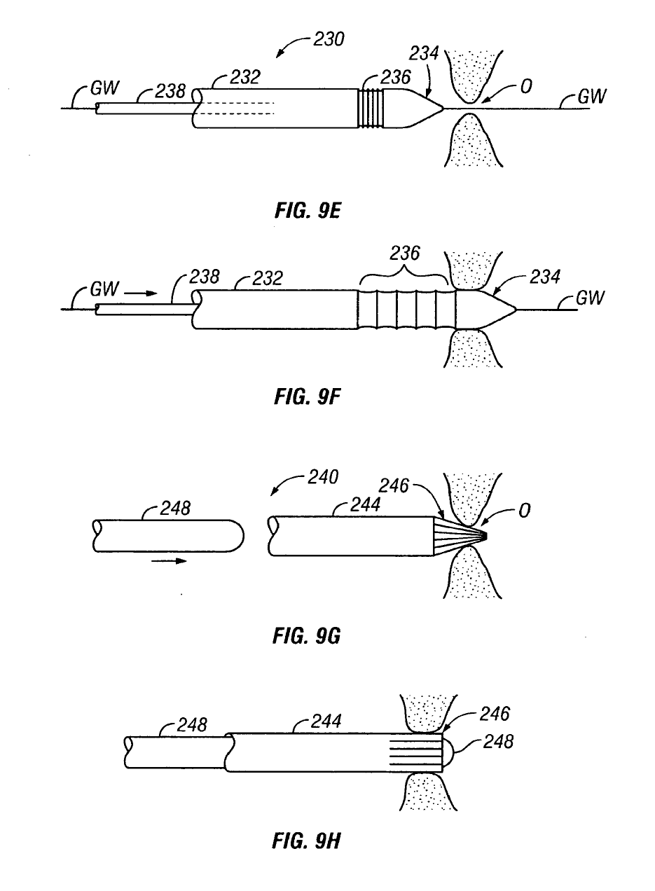

FIGS. 9G and 9H show another dilator device 240. This dilator device 240 comprises an elongate tubular member 244 that has a narrowed or tapered distal end 246. The elongate tubular member 244 has an outer diameter that is larger than the ostium 0 (or other passageway) to be dilated. The narrowed or tapered distal end 246 is small enough to be initially advanced into the ostium 0 (or other passageway) as shown in FIG. 9G. Thereafter, a blunt tipped mandril 248 is advanced though the shaft 244 and into the tapered distal end 246, thereby causing the tapered distal end to expand, resulting in dilation of the ostion 0 (or other passageway) as seen in FIG. 9H.

### Dilators Having Hinged or Pivotal Members

The invention also includes various dilator devices having expandable dilators that comprise hinged or pivotally connected members that move back and forth between a non-expanded configuration and an expanded configuration.

For example, FIGS. 7A and 7B show a dilation device that comprises an expandable dilator 110 mounted on an elongate shaft structure that comprises a flexible, rigid or malleable outer shaft 112 and inner shaft 114. The dilator 110 comprises a number of radially spaced apart rib members 116 that are pivotally connected to a distal portion of the inner shaft 114. Stand off members 118 are pivotally connected at one end to the distal end of the outer shaft member 112 and pivotally connected at the other end to the midregion of each rib member 116. When the dilator is in its non-expanded configuration, the outer shaft member 112 is in a retracted position and the stand off members 118 are lying down generally parallel to the inner shaft member 114. Thereafter, when it is desired to expand the dilator 110, the outer shaft 112 is advanced in the distal direction, causing the stand off members 118 to extend radially outward and substantially perpendicular to the inner shaft member 114, thereby pushing the ribs 116 outwardly to an expanded configuration as seen in FIG. 7B. Optionally, a flexible or elastomeric cover may be disposed on the dilator. Also, optionally, as shown in FIGS. 7A and 7B, a lumen may extend through the inner shaft member 114 and such lumen may be used for advancing the device over a guidewire GW or for other purposes such as irrigation, infusion or aspiration.

FIGS. 7E and 7F show another dilator 110b that may be positioned on a rigid, flexible or malleable shaft. This dilator 110b comprises rigid members 120 and inner deployment members 122 having distal ends that are pivotally connected to the inner surfaced of the rigid members 124 and which have hinges 124 therein. Initially, while the dilator 100b is in its non-expanded configuration, the rigid members 120 are generally parallel to one another. When it is desired to expand the dilator, the inner deployment members are advanced in the distal direction, causing hinges 124 to bend outwardly and resulting in outward spreading of the rigid members 120, as shown in FIG. 7F.

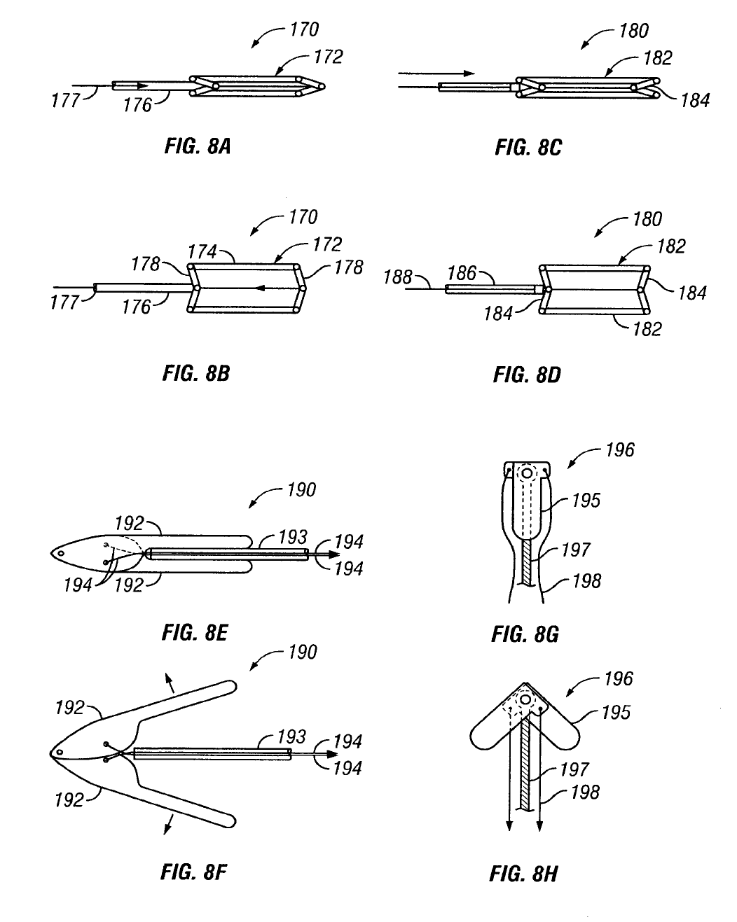

FIGS. 8A and 8B show a dilation device 170 comprising an elongate flexible, rigid or malleable shaft 176 having a hinged dilator 172 on the distal end of the shaft. The hinged dilator 172 comprises rigid side members 174 and hinged end members 178. A pull member 177 extends through shaft 176 and is attached to the distal hinged end member 178. Initially, this hinged dilator 172 is disposed in a non-expanded configuration with both hinged end members 178 collapsed such that their hinges are bent in the same direction, as shown in FIG. 8A. When it is desired to expand the dilator 172, the pull member is retracted in the proximal direction, causing the hinged end members 178 to extends, thereby spreading the rigid side members 174 apart and dilating the passageway within which the dilator 172 is positioned.

FIGS. 8C and 8D show another dilator device 180 comprising an elongate flexible, rigid or malleable shaft 186 having a hinged dilator 182 on the distal end thereof. Initially, this hinged dilator 182 is fixed to the distal end of the shaft in a non-expanded configuration. The hinge or pivot point on the proximal end member 184 is fixed. A hydraulic piston in the shaft 186 is driven by fluid pressure in the distal direction thereby pushing distal hinged end member 184 to an extended position which causes corresponding extension of the fixed hinge or pivot on the proximal end member 184 and, thus, expansion of the dilator as seen in FIG. 8D.

FIGS. 8E and 8F show another dilation device 190 comprising pivoting jaws 192 on the distal end of an elongate flexible, rigid or malleable shaft 193. Cam portions 193 of each jaw 192 are pivotally connected to each other. A separate pull member 194 is attached to each cam portion 195 of each jaw 192. When these pull members 194 are pulled in the proximal direction, the cam portions 195 will partially rotate causing the jaws 192 to open, as seen in FIG. 8F.

FIGS. 8G and 8H show another dilation device 196 wherein pivotally connected arms 195 are attached to the distal end of a shaft 197. Pull members 198 are connected to each arm 195. When the pull members are retracted in the proximal direction, the arms 195 extend outwardly, as seen in FIG. 8H.

### Non-Expandable Tapered Dilators

The invention also includes a number of dilator devices having non-expanding tapered dilators that may be advanced into the desired ostium or other passageway to dilation that ostium or other passageway.

For example, FIG. 9A shows a dilation device 200 which comprises an elongate flexible, rigid or malleable shaft 202 having a tapered distal portion 2004. Optionally a lumen may extend through the device 200 to allow the device 200 to be advanced over a guidewire GW or other uses such as irrigation, infusion or aspiration. The diameter of the shaft 202 is larger than the pre-dilation diameter of the ostium or other passageway to be dilated. Initially, the device 200 is positioned with its distal end within (or in alignment with) the ostium or other passageway to be dilated. The device is then advanced into the ostium or other passageway causing dilation of the ostium or other passageway.

FIG. 9B shows a stepped dilator device 2006 comprising an elongate flexible, rigid or malleable proximal shaft 208 of a first diameter, a proximal tapered region 212, a plateau region 210 of a second diameter that is smaller than the diameter of the shaft 2008 and a distal tapered region 214. Optionally a lumen may extend through the device 206 to allow the device 206 to be advanced over a guidewire GW or for other uses such as irrigation, infusion or aspiration. The diameter of the proximal shaft 208 and/or plateau region 210 is larger than the pre-dilation diameter of the ostium or other passageway to be dilated. Initially, the device 206 is positioned with its distal end within (or in alignment with) the ostium or other passageway to be dilated. The plateau region 210 and/or proximal shaft 208 is/are then advanced into the ostium or other passageway causing dilation of the ostium or other passageway. This device may be useable for dilating more than one passageway of differing size. It will be appreciated that, although the device 2006 shown in this example has only two stepped regions, additional steps may be formed in the device thereby providing additional plateau regions of differing diameter.

FIGS. 9C and 9D show a dilation device 220 comprising an elongate flexible, rigid or malleable shaft 222 having a tapered distal portion 224. Optionally a lumen may extend through the device 220 to allow the device 220 to be advanced over a guidewire GW or for other uses such as irrigation, infusion or aspiration. A frusto-conical everting sheath 226 formed of smooth material (e.g., a lubricious polymer) is initially attached to the distal end of the device 220 as shown in FIG. 9C. The diameter of the shaft 220 is larger than the pre-dilation diameter of the ostium or other passageway to be dilated. Initially, the device 220 is positioned with its distal end within (or in alignment with) the ostium or other passageway to be dilated. The device 220 is then advanced into the ostium 0 or other passageway causing the sheath 226 to evert over the tapered distal portion 224 of the device, thereby facilitation smooth advancement of the tapered distal portion into the ostium 0 or other passageway and thereby causing dilation of the ostium 0 or other passageway, as seen in FIG. 9.

FIGS. 9E and 9F show another dilation device 230 comprising an elongate flexible, rigid or malleable shaft 232 having a push member 238 therein, a tapered distal portion 234 and telescoping region 236. Optionally a lumen may extend through the device 200 to allow the device 200 to be advanced over a guidewire GW or for other uses such as irrigation, infusion or aspiration. The diameter of the shaft 232 is larger than the pre-dilation diameter of the ostium 0 or other passageway to be dilated. Initially, the device 232 is positioned with its distal end within (or in alignment with) the ostium 0 or other passageway to be dilated. Thereafter, the push member 238 is advanced, causing the telescoping region 236 to extend telescopically and causing the tapered distal portion 234 to advance into or through the ostium 0 or other passageway thereby resulting in the desired dilation.

FIG. 9I shows another dilation device 250 comprising an elongate rigid or malleable shaft 252 having a bulbous dilator 256 at the distal end of the shaft 252 and a handle 260 on the proximal end of the shaft 252. Optionally a lumen may extend through the device 250 to allow the device 250 to be advanced over a guidewire GW or for other uses such as irrigation, infusion or aspiration. The diameter of the bulbous dilator 256 is larger than the pre-dilation diameter of the ostium or other passageway to be dilated. Initially, the device 250 is positioned with the bulbous dilator 256 adjacent to the ostium or other passageway to be dilated. Thereafter, the device 250 is further advanced causing the bulbous dilator 256 to dilate the ostium or other passageway.

FIG. 9J shows similar dilation device that is of the same construction as the device of FIG. 91 except that its shaft 250a is curved. This curved shaft may be useful for accessing certain ostia or passageways of the ear, nose or throat, such as the frontal or maxillary sinuses or the Eustachian tube.

### Dilators that Expand in Response to Conditions within the Body

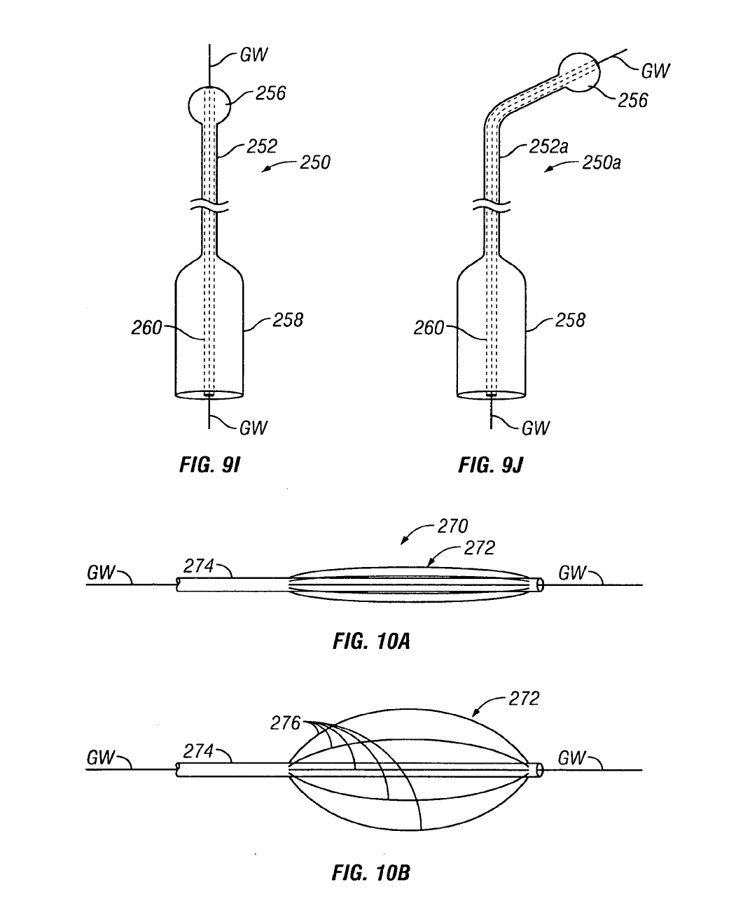

The present invention also includes dilator devices that have dilators that expand in response to certain conditions present within the ostium or other passageway to be dilated. For example, FIGS. 10A and 10B show a dilation device 270 comprising a flexible, rigid or malleable shaft 294 having a dilator 272 thereon. This dilator 272 comprises a plurality of struts 276, both ends of which are secured to the shaft 279. These struts 276 are formed of a shape memory material, such as nickel-titanium alloy, which elongate and change shape causing the dilator 272 to expand as it warms to body temperature. Initially, at room temperature, the dilator 272 has the non-expanded configuration shown in FIG. 10A. After the dilator has been positioned within the ostium or other passageway, it warms to body temperature, thereby causing the dilator 272 to expand as seen in FIG. 10B and thus dilating the ostium or other passageway. Optionally a lumen may extend through the device 270 to allow the device 270 to be advanced.

### Mechanical Dilators in Combination with Balloons

In some embodiments of devices of the present invention, a balloon may be provided in addition to a mechanical dilator. Such devices may be particularly useful in cases where some initial dilation of a passageway is best performed using a mechanical dilator, but further dilation of the passageway is best performed using a balloon.

FIGS. 12A and 12B show an example of a device 300 that has a rigid, flexible or malleable shaft 310 and both a mechanical dilator 302 and a balloon 304 on the shaft 304. The shaft 310 comprises an outer tube 310 and an inner tube 312 which extends through and beyond the distal end of the outer tube 310. The mechanical dilator 302 comprises rigid side members 306 which are on opposite sides of and aligned with the inner tube 312, as shown. The rigid side members 306 are as wide or slightly wider than the inner tube 312. Right proximal and distal hinged end members 308R connect the right sides of the rigid side members 306. Left proximal and distal hinged end members 308L connect the left sides of the rigid side members 306. A tapered bushing 314 surrounds and is in fixed position on the inner shaft 312. Right and left pull member guides holes extend longitudinally though the right and left sides of fixed bushing 314. A balloon 304 is mounted on the device such that it fully surrounds the mechanical dilator 302. This balloon may comprise a compliant, semicompliant or noncompliant balloon. For some applications where the ostia or paranasal sinuses are to be dilated, this balloon may be a non-compliant polyethylene teraphthalate (PET) (balloon capable of being inflated to approximately 14 atmospheres. When the device is in its non-expanded state, the hinged end members 308L, 308R will be collapsed and the proximal hinged end members 308R and 308L may rest against the tapered surface of fixed bushing 314 and the balloon 304 will be deflated. After the dilator 300 has been positioned within the ostium or passageway to be dilated, the right and left members 316R, 316L are pulled in the proximal direction, causing the hinged end members 308R, 308L to extend, thereby spreading rigid side members 306 apart. The rigid side members will exert pressure on the surrounding ostium or passageway, thereby dilating it an initial amount or causing an initial effect (e.g., breaking or remodeling bone). Thereafter, the balloon 304 may be inflated to further dilate the ostium or passageway to an even larger diameter and/or to a different shape and/or to produce a different effect (e.g., soft tissue compression, hemostasis, etc) than had been achieved by the initial dilation using the mechanical dilator 302. After the dilation is complete, the balloon may be deflated and members 316R, 316L may in some cases be pushed in the distal direction to assist in collapsing of the mechanical dilator assembly 302.

Although FIGS. 12A and 12B show a balloon 304 that surrounds a mechanical dilator 302, it is to be appreciated that the balloon 304 may alternatively be located elsewhere on the shaft (e.g., proximal or distal to the mechanical dilator) and the device may be advanced or retracted as needed to alternately position the balloon 304 and mechanical dilator 302. Or, the balloon 304 may be proximal or distal to the mechanical dilator 302 and one may be used to dilate a region of a passageway within which it is positioned and the other may be used to dilate a different region of the passageway within which it is positioned.

It is to be appreciated that the invention has been described hereabove with reference to certain examples or embodiments of the invention but that various additions, deletions, alterations and modifications may be made to those examples and embodiments without departing from the intended spirit and scope of the invention. For example, any element or attribute of one embodiment or example may be incorporated into or used with another embodiment or example, unless otherwise specified of if to do so would render the embodiment or example unsuitable for its intended use. Also, where the steps of a method or process have been described or listed in a particular order, the order of such steps may be changed unless otherwise specified or unless doing so would render the method or process unworkable for its intended purpose. All reasonable additions, deletions, modifications and alterations are to be considered equivalents of the described examples and embodiments and are to be included within the scope of the following claims.

1.-28. (canceled)

29. A method for dilating a passageway in a head of a subject using a dilation device, wherein the dilation device comprises an elongate member extending along a longitudinal axis and a dilator comprising a pivoting member, wherein the pivoting member is configured to pivot relative to the longitudinal axis in response to movement of the elongate member, wherein the dilator is configured to expand in response to pivotal movement of the pivoting member relative to the longitudinal axis, wherein the method comprises:
(a) inserting the dilation device into a nostril of the subject,
(b) positioning the dilator in or adjacent to a passageway in the head of the subject; and
(c) moving the elongate member to cause the pivoting member to pivot such that the dilator expands and dilates the passageway.

30. The method according to claim 29, wherein the dilation device includes a proximal end and a distal end, wherein the method further comprises moving the elongate member toward the distal end to cause the pivoting member to pivot.

31. The method according to claim 29, wherein the dilation device includes a proximal end and a distal end, wherein the method further comprises moving the elongate member toward the proximal end to cause the pivoting member to pivot.

32. The method according to claim 29, wherein the act of moving the elongate member pivots the pivoting member away from the longitudinal axis, wherein the dilator expands in response to pivoting of the pivoting member away from the longitudinal axis

33. The method according to claim 29, wherein the elongate member comprises an inner shaft and an outer shaft, wherein the pivoting member comprises a plurality of ribs pivotally connected to an outer shaft, wherein the dilator further comprises a cover operably coupled to the ribs, wherein moving the elongate member comprises advancing the outer shaft in a distal direction, thereby causing the ribs to pivot outwardly away from the longitudinal shaft.

34. The method according to claim 29, wherein the elongate member comprises a pair of inner deployment members operably coupled to the dilator, wherein the inner deployment members include distal ends that are pivotally connected via hinges to respective rigid members of the dilator, wherein moving the elongate member comprises advancing the inner deployment members in the distal direction, thereby causing rigid members to move radially outwardly from the longitudinal axis and thereby dilate the passageway.

35. The method according to claim 29, wherein the pivoting member comprises a first rigid side member, a second rigid side member, a proximal hinged end member between the first and second rigid side members, and a distal hinged end member between the first and second rigid side members and opposing the proximal hinged end member, wherein the distal hinged end member is coupled to the elongate member.

36. The method according to claim 35, wherein moving the elongate member comprises moving the elongate member in a proximal direction, thereby moving the distal hinged member in the proximal direction and causing the first and second rigid side members to move radially outwardly from the longitudinal axis and thereby dilate the passageway.

37. The method according to claim 29, wherein the pivoting member further comprises a pair of jaws, wherein the elongate member comprises a pair of pull members coupled to the jaws, and the method further comprises pulling the pull members in a proximal direction to open the jaws away from the longitudinal axis and thereby dilate the passageway.

38. The method according to claim 37, wherein the jaws each comprise a cam member, wherein the cam members are pivotally connected to one another, wherein each of the pull members is pivotally connected to a respective cam member.

39. The method according to claim 29, wherein the dilator further comprises an expandable balloon, wherein the method further comprises expanding the expandable balloon to dilate the passageway.

40. The method according to claim 29, wherein the passageway is an opening in a paranasal sinus.

41. The method according to claim 29, further comprising inserting a guide catheter into the nostril of the subject.

42. The method according to claim 29, further comprising inserting a guidewire into the nostril of the subject.

43. The method according to claim 29, further comprising inserting an endoscope into the nostril of the subject.

44. A method for dilating a passageway in a head of a subject using a dilation device, wherein the dilation device comprises an elongate member extending along a longitudinal axis and a spreading member moveable in response to movement of the elongate member, wherein the dilation device further comprises a dilator, wherein the dilator is configured to expand transversely to the longitudinal axis in response to movement of the spreading member, wherein the method comprises:
(a) inserting the dilation device into a nostril of the subject;
(b) positioning the dilator in or adjacent to a passageway in the head of the subject; and
(c) moving the elongate member to cause movement of the spreading member, thereby expanding the dilator to dilate the passageway.

45. The method according to claim 44, wherein at least one of the spreading member or the dilator comprises a tapered region, wherein the tapered region causes the dilator to move transversely relative to the longitudinal axis in response to movement of the spreading member.

46. The method according to claim 45, wherein the tapered region is disposed on the spreading member, wherein the method further comprises moving the elongate member to cause movement of the spreading member, wherein the dilator cams radially outwardly along the spreading member in response to movement of the spreading member.

47. The method according to claim 44, wherein the spreading member comprises a wedge, wherein the dilator comprises a first portion and a second portion, wherein the method comprises moving the elongate member to cause movement of the spreading member such that the wedge is moved to a position between the first portion and the second portion, thereby spreading the first portion and second portion away from each other and the longitudinal axis.

48. A method for dilating a passageway in a head of a subject using a dilation device, wherein the dilation device comprises a shaft and a dilator positioned on the shaft, wherein the dilator comprises at least one cam member covered by an expandable cover, wherein the cover is configured to expand in response to activation of the at least one cam member, wherein the method comprises:
(a) inserting the dilation device into a nostril of the subject,
(b) positioning the dilator in or adjacent to a passageway in the head of the subject; and
(c) activating the at least one cam member to cause expansion of the expandable cover, thereby dilating the passageway.
